(19) United States
(12) Patent Application Publication
(10) Pub. No.: US 2003/0120180 A1
(43) Pub. Date: Jun. 26, 2003

(54) Method and apparatus for collecting and testing biological samples

(75) Inventors: Rosann Marie Matthews Kaylor, Cumming, GA (US); Dennis Stein Everhart, Alpharetta, GA (US); Jeffrey Dean Lindsay, Appleton, WI (US); Jason Patrick McDevitt, Alpharetta, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/027,265

(22) Filed: Dec. 21, 2001

(51) Int. Cl. A61B 5/00

(52) U.S. Cl. 600/584

(57) ABSTRACT

A device for capturing a substance, the device including a generally tubular body including a generally tubular inner surface defined by a first layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface generally disposed radially outwardly from the inner surface, wherein a portion of the outer surface is adapted to capture the substance. Also, a method for collecting a sample from a test subject, the method including providing a device adapted to capture and retain the sample, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface; inserting a finger into the pocket; and contacting the substance to be sampled with the device.

### BACKGROUND

Cotton balls, swabs, and gauzes are commonly used for a variety of reasons. For instance, a cotton ball can be used to apply diaper rash ointments, medications, alcohol, oral anesthetics, etc. In each of these fields, the cotton ball or swab is typically configured to deliver a particular additive or ingredient to the area of application.

Several commercial products exist for biological sample collection, including simple cotton swabs or more recent products such as the SALIVETTE-brand saliva collecting tube by Sarstedt International, which has a cotton swab or roll inside the tube. Another swab-based device made from plastic, porous materials is described in Japanese Patent No. JP 63215939. Other saliva collection devices are the OMNI-SAL-brand collection device, made by Saliva Diagnostic Systems, Inc., and Epitope's ORASURE-brand oral specimen collection device. Another form of a sample collection diagnostic product is the HEMAWIPE-brand collection system by MedTek Diagnostics, which involves an absorbent pad that a patient uses to wipe after a bowel movement. The patient then mails the pad back to the physician for analysis of fecal occult blood.

PCT Patent Nos. WO 9609544 and WO 9609545 describe an elongated sampling element suitable for insertion, such as into the vagina to collect cervical mucous for analysis of fertility. Patents such as PCT Patent Nos. WO 0072009 and WO 9535497 describe absorbent materials to collect body fluids; these absorbent devices use hydrophilic materials such as cotton to collect the sample.

### SUMMARY OF THE INVENTION

A significant problem faced by a user of currently-available sample collection or application devices lies in the exposure of the user to the sample. For example, the literature describes most of the over-the-counter fecal occult blood tests as “unpleasant to use” due to specimen collection (see Diagnostics Intelligence, vol. 10, no. 9, p.13). An additional problem lies in the difficulty, in some instances, for a user to apply an additive to a cotton ball, for example, without undesirably spilling some of the additive. Moreover, cotton materials can often be relatively expensive and difficult to process in comparison to other types of materials. Additionally, in many cases, biologically relevant samples or biomarkers are proteins, which are known to adsorb preferentially to hydrophobic materials. Thus, the use of absorbent materials could result in a dilution of the desired protein, because these absorbent devices would tend to preferentially absorb the surrounding matrix fluid rather than the analyte of interest.

As such, a need currently exists for an improved product capable of collecting a sample, or of delivering an additive, such as an indicator, to a particular area of application. In particular, a need currently exists for a finger glove device capable of insulating a finger while collecting a sample or delivering a particular additive.

This invention describes a device for capturing a substance, the device including a generally tubular body including a generally tubular inner surface defined by a first layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface generally disposed radially outwardly from the inner surface, wherein a portion of the outer surface is adapted to capture the substance. Also, a method for collecting a sample from a test subject, the method including providing a device adapted to capture and retain the sample, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface; inserting a finger into the pocket; and contacting the substance to be sampled with the device.

A key advantage that the proposed device offers for the user is isolation and protection from getting in contact with body fluids. Swabs, wipes, or other such collection devices do not offer this same level of protection. Another advantage to the proposed device is the dexterity offered by still being able to use one's finger rather than manipulate a swab; this dexterity allows better sample collection in some cases.

Other objects and advantages of the present invention will become more apparent to those skilled in the art in view of the following description and the accompanying drawings.

### BRIEF DESCRIPTION OF THE DRAWINGS

A full and enabling disclosure of the present invention, including the best mode thereof, directed to one of ordinary skill in the art, is set forth in the specification, which makes reference to the appended drawings.

FIG. 1 is a perspective view of a device on a finger according to one embodiment of the present invention.

FIG. 2 is a perspective view of a two-sided device according to one embodiment of the present invention.

FIG. 3 is a perspective view of a bottom section of a two-sided device according to one embodiment of the present invention.

FIG. 4 is a perspective view of the bottom section of the two-sided device of FIG. 3 attached to a top section to form the two-sided device according to one embodiment of the present invention.

FIG. 5 is a perspective view of a device turned “inside-out” according to one embodiment of the present invention.

FIG. 6 is a perspective view of a device turned “inside-out” according to another embodiment of the present invention.

FIG. 7 is a perspective view of a device having a safety mechanism according to one embodiment of the present invention.

FIG. 8 is a perspective view of an embodiment of a device having a unitary structure.

FIG. 9 is a perspective view of a tapered device having two open ends according to one embodiment of the present invention.

FIG. 10 is a perspective view with cut away portions illustrating one embodiment of a method for turning a device of the present invention inside-out.

Repeat use of reference characters in the present specification and drawings is intended to represent the same or analogous features or elements of the invention.

### DETAILED DESCRIPTION OF THE PREFERRED EMBODIMENTS

As used herein, the term “barrier material” refers to a material that is substantially impermeable to chemical liquids and solids, including any bodily fluids and contaminants, as well as to biological particles such as viruses, bacteria, or other pathogens. The barrier material may be a blown, cast, or extruded sheet of thermoplastic polymer, free of pores, for example, or other material as described herein.

As used herein, the term “biconstituent fibers” refers to fibers that have been formed from at least two polymers extruded from the same extruder as a blend. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes referred to as multiconstituent fibers. Fibers of this general type are discussed in, for example, U.S. Pat. Nos. 5,108,827 and 5,294,482 to Gessner. Biconstituent fibers are also discussed in the textbook Polymer Blends and Composites by J. A. Manson and L. H. Sperling, 1976, at pages 273-77.

As used herein, the term “breathable” means pervious to water vapor and gases. In other words, “breathable barriers” and “breathable films” allow water vapor to pass therethrough, but are substantially impervious to liquid water. For example, “breathable” can refer to a film or laminate having water vapor transmission rate (WVTR) of at least about 300g/m² /24 hours measured using ASTM Standard E96-80, upright cup method, with minor variations as described in the following Test Procedure.

A measure of the breathability of a fabric is the water vapor transmission rate (WVTR) which, for sample materials, is calculated essentially in accordance with ASTM Standard E96-80 with minor variations in test procedure as set forth herein. Circular samples measuring three inches in diameter are cut from each of the test materials, and tested along with a control, which is a piece of ACELGARD@ 2500 sheet from Celanese Separation Products of Charlotte, N.C. ACELGARD@ 2500 sheet is a microporous polypropylene sheet. Three samples are prepared for each material. The test dish is a No. 60-1 VAPOMETER-brand pan distributed by Thwing-Albert Instrument Company of Philadelphia, Pa. 100 milliliters of water is poured into each and individual samples of the test materials and control material are placed across the open tops of the individual pans. Screw-on flanges are tightened to form a seal along the edges of the pan, leaving the associated test material or control material exposed to the ambient atmosphere over a 6.5 cm diameter circle having an exposed area of approximately 33.17 square centimeters. The pans are placed in a forced air oven at 100° F. (32° C.) for one hour to equilibrate. The oven is a constant temperature oven with external air circulating through it to prevent water vapor accumulation inside. A suitable forced air oven is, for example, a BLUE M POWER-O-MATIC 600-brand oven distributed by Blue M Electric Company of Blue Island, Ill. Upon completion of the equilibration, the pans are removed from the oven, weighed and immediately returned to the oven. After 24 hours, the pans are removed from the oven and weighed again. The preliminary test water vapor transmission rate values are calculated as follows: Test WVTR=(grams weight loss over 24 hours)×(315.5 g/m² /24 hours).

The relative humidity within the oven is not specifically controlled. Under predetermined set conditions of 100° F. (32° C.) and ambient relative humidity, the WVTR for the ACELGARD@ 2500 control has been defined to be 5000 grams per square meter for 24 hours. Accordingly, the control sample was run with each test and the preliminary test values were corrected to set conditions using the following equation: WVTR=(test WVTR/control WVTR)×(5000 g/m² /24 hrs.).

As used herein, the term “conjugate fibers” refers to fibers which have been formed from at least two polymers extruded from separated extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement, wherein one polymer is surrounded by another or may be a side by side arrangement, a pie arrangement or an “islands-in-the-sea” arrangement. Conjugate fibers are taught by U.S. Pat. Nos. 5,108,820 to Kaneko et al., 4,795,668 to Krueger et al., and 5,336,552 to Strack et al. Conjugate fibers are also taught in U.S. Pat. No. 5,382,400 to Pike et al. and may be used to produced crimp in the fibers by using the differential rates of expansion and contraction of the two (or more) polymers. Crimped fibers may also be produced by mechanical means and by the process of German Patent DT 25 13 251 A1. For two component fibers, the polymers may be present in ratios of 75/25, 50/50, 25/75, or any other desired ratios. The fibers may also have shapes such as those described in U.S. Pat. Nos. 5,277,976 to Hogle et al., 5,466,410 to Hill, 5,069,970 to Largman et al., and 5,057,368 to Largman et al., which describe fibers with unconventional shapes.

As used herein, the terms “elastic” and “elastomeric” are generally used to refer to materials that, upon application of a force, are stretchable to a stretched, biased length which is at least about 133%, or one and a third times, its relaxed, unstretched length, and which will recover at least about 50% of its elongation upon release of the stretching, biasing force.

As used herein, the term “filament” refers to a generally continuous strand that has a large ratio of length to diameter, such as, for example, a ratio of 1000 or more.

As used herein, “meltblown fibers” refers to fibers formed by extruding a molten thermoplastic material through a plurality of fine, usually circular, die capillaries as molten threads or filaments into converging high velocity, usually hot, gas (e.g. air) streams which attenuate the filaments of thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al.. Meltblown fibers are microfibers that may be continuous or discontinuous, are generally smaller than 10 microns in average diameter, and are generally tacky when deposited on a collecting surface.

As used herein, a “moisture barrier” refers to any material that is relatively impermeable to the transmission of fluids, i.e. a fabric having a moisture barrier can have a blood strikethrough ratio of 1.0 or less according to ASTM test method 22.

As used herein, the term “nonwoven web” refers to a web having a structure of individual fibers or threads that are interlaid, but not in an identifiable manner as in a knitted fabric. Nonwoven webs or fabrics have been formed from many processes, such as, for example, meltblowing processes, spunbonding processes, and bonded carded web processes. The basis weight of nonwoven fabrics is usually expressed in ounces of material per square yard (osy) or grams per square meter (gsm) and the fibers diameters are usually expressed in microns. (Note that to convert from osy to gsm, multiply osy by 33.91).

As used herein, “spunbond fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. Nos. 4,340,563 to Appel et al., 3,692,618 to Dorschner et al., 3,802,817 to Matsuki et al., 3,338,992 to Kinney, 3,341,394 to Kinney, 3,502,763 to Hartman, and 3,542,615 to Dobo et al.. Spunbond fibers are generally not tacky when they are deposited on a collecting surface. Spunbond fibers are generally continuous and have average diameters (from a sample of at least 10) larger than 7 microns, and more particularly, between about 10 and 40 microns.

As used herein, the term “substance” is used to generally describe any sort of sample, agent, or additive that may be used by or collected in conjunction with a device. Substance may refer to any liquid, solid, or semi-solid that can be collected, retained, released, absorbed, adsorbed, or applied to, by, or from the device.

As used herein, the term “texturized” refers to a base web having projections from a surface of the web in the Z-direction. The projections can have a length, for instance, from about 0.1 mm to about 25 mm, particularly from about 0.1 mm to about 5 mm, and more particularly from about 0.1 mm to about 3 mm. The projections can take on many forms and can be, for instance, bristles, tufts, loop structures such as the loops used in hook and loop attachment structures, and the like.

FIGS. 1-10 illustrate an apparatus and a method to collect samples from a body for subsequent diagnostic testing, or to apply a substance to the body. The primary device is a finger-glove-type device, which is in a generally tubular shape such that it fits snugly over the finger of a user. The device is essentially similar to the devices described in co-pending U.S. patent applications Ser. No. 09/826,413, “Finger Glove,” filed Apr. 4, 2001, Ser. No. 09/826,371, “Disposable Finger Sleeve for Appendages,” filed Apr. 4, 2001, and Ser. No. 09/826,411, “Dental Wipes,” filed Apr. 4, 2001, which are incorporated herein by reference. The preferred embodiments are described herein, although variations on the finger-glove-type device are contemplated, included variations with zero, one, or more seams, and any other suitable combinations of materials.

The device includes a nonwoven exterior to efficiently collect and subsequently release the body fluid or sample, and has a protective barrier material on its interior to hygienically protect the user from such fluids or samples.

More specifically, devices made in accordance with the present invention are generally constructed from disposable materials, such as nonwoven webs made from synthetic and/or pulp fibers. Further, the device can also include an elastic component for providing the glove with form-fitting properties.

For instance, it has been discovered that by forming a device with an elastic nonwoven material in accordance with the present invention, the resulting glove can snugly fit onto a person's finger so that the glove can more effectively remain on the finger throughout use. Moreover, a device of the present invention can remain breathable to aid in a person's comfort during use, while also remaining capable of substantially inhibiting the transfer of liquids from the outer surface of the glove to the person's finger.

A device of the present invention can generally be formed in a variety of ways. For instance, in one embodiment, the device can be formed as a unitary structure from a particular base web material, such as an elastomeric nonwoven base web material. Moreover, in another embodiment of the present invention, the device can be formed from two or more sections of base web material. Each section can be identical or different, depending on the desired characteristics of the device. For example, in one embodiment, the device is formed from two sections, wherein one section is formed from a textured nonwoven material and the other section is formed from an elastomeric nonwoven material.

Referring to FIGS. 1-9, various embodiments of devices made in accordance with the present invention are depicted. In general, a device of the present invention can be used to apply medications, ointments, indicator agents, etc., or to remove substances, e.g., for sample taking or make-up removal. For example, in one embodiment, as shown in FIG. 1, a device 10 can be placed over a finger 11 for collecting a sample or for applying a substance. In each embodiment, the outwardly-facing layer is also known as the exterior layer, having an outer surface, and the inwardly-facing layer is also known as the interior layer, having an inner surface, whether or not a particular embodiment is turned inside-out.

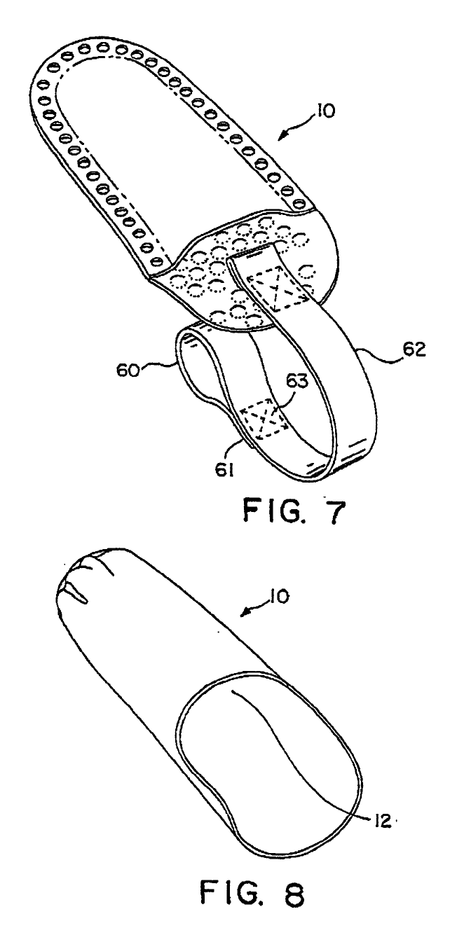

One embodiment of a device of the present invention is depicted in FIG. 8. As shown, the device 10 is formed as a unitary structure from a single piece of fabric.

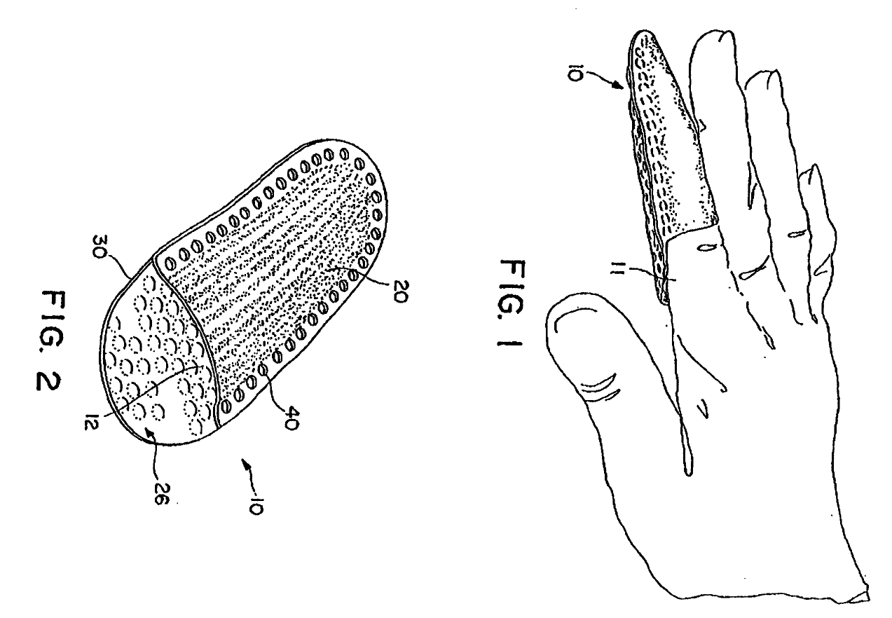

Referring to FIGS. 2-4, another embodiment of a device of the present invention is depicted. As shown, instead of a unitary structure, the device 10 is made from a first section 20 and a second section 30. Generally, one section of the device 10 can be bonded or attached to the other section in a finger-shaped pattern by any suitable manner, such as by adhesive, thermal, or mechanical bonding, so that the connection of the sections can form a pocket 12 for the insertion of a finger. In the embodiment depicted in FIG. 2, for example, the first section 20 is attached in a finger-shaped pattern to the second section 30 at their respective outer edges via the seams 40 to form a device 10 having a pocket 12. Once each section is bonded or attached at the seams 40, the materials forming each of the sections 20 and 30 can then be cut adjacent to the seams such that the finger-shaped glove 10 is formed.

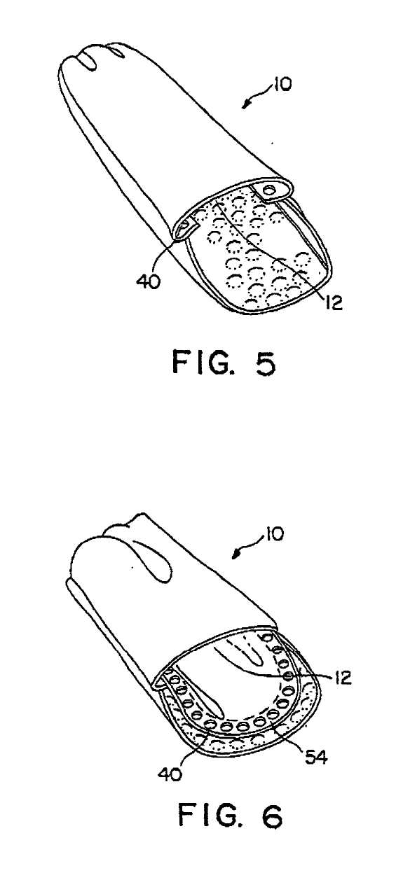

In some embodiments, as depicted in FIGS. 5-6, the device 10 can also be turned inside-out such that the seams 40 are located inside the pocket 12. For example, as shown in FIG. 6, the seams 40 of the device 10 can be pushed into the pocket 12 such that the seams 40 remain on the inside of the glove 10, as depicted in FIG. 5. This inside-out position, as shown in FIG. 5, can provide the device with improved aesthetics. Moreover, the seams in the inside-out position can also provide a better fit by providing more friction applied to the finger. In addition, in some embodiments, this inside-out position can enable the glove 10 to be more resistant to flattening during use.

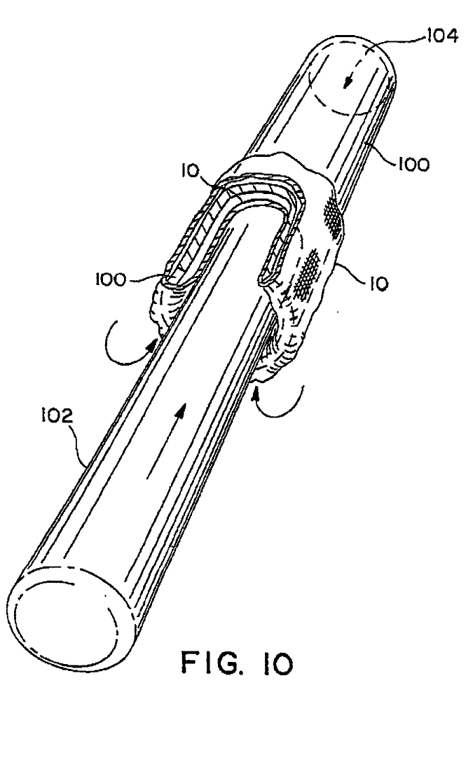

Various methods can be used in order to place the device in the inside-out position. For instance, the device can be turned inside-out using a pressurized gas, a vacuum, or mechanical means. For example, one mechanical method for inverting the device is illustrated in FIG. 10. As shown, in this embodiment, the device 10 is placed over a cylinder 100. The cylinder 100 defines a passage 104. In order to invert the device 10, a rod 102, preferably with a compressible tip, is used to push against the closed end of the device until the device is pushed all the way through the passage 104. Through this process, the device 10 is inverted.

As shown in FIGS. 1-4, the first section 20 can, in some embodiments, have a length greater than the second section 30 such that first section 20 includes a portion or pull-on tab 26 that extends beyond the edge of the second section 30. By extending beyond the second section 30, the portion 26 can facilitate placement of a device 10 over a finger. In particular, a user can conveniently grab the portion 26 to place the device 10 over a finger. Besides the first section 20, a pull-on tab can be positioned on any suitable portion of the device. For instance, the pull-on tab can be located on the second section also.

Further, in another embodiment, the pull-on tab 26 can also be provided in the middle portion of the device 10 such that a user can the pull the tab 26 in a direction perpendicular to the lengthwise direction of a flattened device. As a result, the tab 26 can facilitate the insertion of a finger into the glove 10 by “spreading out” the glove in an upwardly direction as a finger is inserted therein.

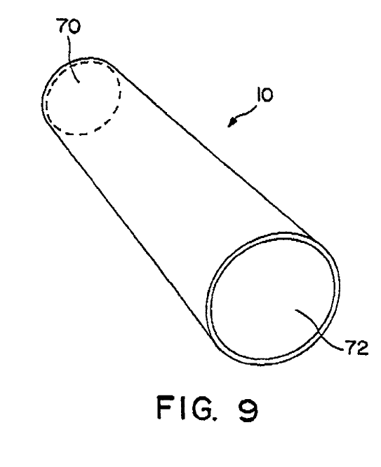

Further, in other embodiments, as shown in FIG. 9, a device 10 can also be provided with a tapered shape to enhance the ability of the glove to fit onto a finger. In addition, as shown in FIG. 9, a glove 10 can have two open ends 70, 72 so that a finger can be inserted completely therethrough.

In some embodiments of the present invention, it may also be desirable to provide the device 10 with certain safety features. In particular, although a device of the present invention can fit tightly onto a finger, a safety mechanism can help ensure that the glove does not fall into or remain behind in a user's orifice during use. Specifically, a safety mechanism of the present invention can attach to one finger of a user, while the device is fitted onto another finger. For example, as shown in FIG. 7, one embodiment of a safety mechanism of the present invention includes a safety portion 60 that can fit around a finger of a user.

In one embodiment of the present invention, as shown in FIG. 7, the safety mechanism can also include a linking portion 62 for attaching the safety portion 60 to the device 10. When utilized, the linking portion 62 can be attached to the glove using a variety of well-known attachment methods, such as thermal, chemical, or mechanical bonding. For example, in one embodiment, the linking portion 62 is attached to a device 10 by an adhesive. In another embodiment, the linking portion 62 is attached to a glove 10 by stitching.

In general, the linking portion 62 can be made from a variety of materials, such as strings, bands, cords, fibers, nonwovens, etc. For most applications, the linking portion 62 can have a length of from about 1 inch to about 12 inches. In general, the safety portion 60 can have any shape as long the shaped safety portion can fit on the finger. For instance, in the embodiment shown in FIG. 7, a safety portion 60 is formed to have a loop or ring shape such that it can be secured on a finger. Moreover, the safety portion can also be formed into a certain shape from the linking portion itself. For instance, as shown in FIG. 7, an end 61 of a linking portion 62 is folded and attached to the portion 63 of the linking portion 62 via stitching to form a ring-shaped safety portion 60. It should be understood that the end 61 can also be attached to a portion 63 by any attachment method known in the art, such as, for example, thermal, chemical, or mechanical bonding methods. Although not specifically depicted, the safety portion 60 can also be formed from a material separate from the linking portion 62. When formed separately, the safety portion 60 can be attached to linking the portion 60 by any attachment method known in the art, such as, for example, thermal, chemical, or mechanical bonding methods.

Typically, the safety portion can be made from the same or a different material than the linking portion. For example, in one embodiment, a safety portion 60 contains an elastic material, such as an elastomeric nonwoven, which can allow the safety portion to fit more tightly around a finger.

As shown in FIG. 7, when the linking portion 62 is utilized to attach safety portion 60 to the device 10, the safety mechanism of the present invention can effectively prevent the glove 10 from falling off.

In general, the device of the present invention, such as depicted in FIGS. 1-9, can be formed from a variety of materials. For instance, in one embodiment, the first section 20 and the second section 30 are formed from a base web. It should be understood, however, that, as used herein, a base web of the present invention is meant to include one or more layers of fibrous materials. Generally, a device made according to the present invention can be made from any suitable material used for making wipes.

For most applications, devices made in accordance with the present invention are constructed from nonwoven webs containing an elastic component. The elastic component, for instance, can form a separate section of the device. For example, the device can be made from two or more sections of material that includes a first section made from a non-elastic material and a second section made from an elastic material.

Alternatively, the device can be made from a single piece of material that contains an elastic component. For example, in this embodiment, the elastic component can be a film, strands, nonwoven webs, or elastic filament incorporated into a laminate structure.

Non-elastic materials used in the present invention typically include nonwoven webs or films. The nonwoven webs, for instance, can be melt-blown webs, spunbond webs, carded webs, and the like. The webs can be made from various fibers, such as synthetic or natural fibers.

For instance, in one embodiment, synthetic fibers, such as fibers made from thermoplastic polymers, can be used to construct the device of the present invention. For example, suitable fibers could include melt-spun filaments, staple fibers, melt-spun multi-component filaments, and the like.

These synthetic fibers or filaments used in making the nonwoven material of the base web may have any suitable morphology and may include hollow or solid, straight or crimped, single component, conjugate or biconstituent fibers or filaments, and blends or mixtures of such fibers and/or filaments, as are well known in the art.

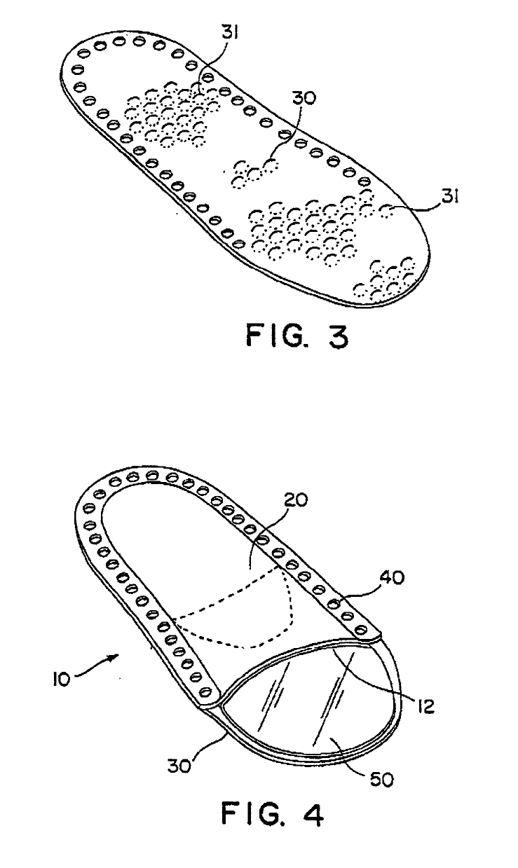

Besides including a non-elastic component or an elastic component, the device of the present invention can further include a barrier material that is incorporated into or laminated to a base web of the present invention. When such a barrier material is a moisture barrier, the barrier can prevent, or at least minimize, leakage from outside the glove by establishing a barrier to the passage of liquid from the glove to the finger placed therein. For example, as shown in FIG. 4, a layer of material or film can be provided to form the barrier material 50, which can act as a barrier between the outer layer of the glove 10 and a finger. Moreover, in this embodiment, as shown in FIG. 4, a barrier material 50 can act as an inner lining for the second section 30 only, while the first section 20 possesses no such inner lining. However, it should also be understood that the barrier material 50 may be a liner for both the first section 20 and the second section 30. Moreover, the barrier material 50 can be applied asymmetrically or unevenly to the glove such that one portion of the glove is substantially moisture impervious, while another portion is not. It should be understood that a barrier material 50 can be applied to the glove 10 as a layer of the base web, or as an outer lining for the base web. Moreover, it should also be understood that the barrier material can be inherent within the base web structure such that it would not constitute a separate lining thereof.

The barrier material may be chosen to be substantially impermeable to one or more of chemical or other liquids and solids, including any bodily fluids and contaminants, as well as to biological particles such as viruses, bacteria, or other pathogens.

In one embodiment of the present invention, the barrier material 50 can be made from liquid-impermeable plastic films, such as polyethylene and polypropylene films. Generally, such plastic films are impermeable to gases and water vapor, as well as liquids.

While completely liquid-impermeable films can prevent the migration of liquid from outside the glove to the finger, the use of such liquid- and vapor-impermeable barriers can sometimes result in a relatively uncomfortable level of humidity being maintained in a glove 10.

As such, in some embodiments, breathable, liquid-impermeable barriers are desired. A moisture barrier can act as a barrier material as described herein. Also, moisture barrier layers, as described above, can be used alone or incorporated into a laminate when used to construct the device of the present invention. When incorporated into a laminate, the laminate can include various nonwoven webs in combination with the moisture barrier layer. For instance, moisture barrier laminates can be formed from many processes, such as for example, meltblowing processes, spunbonding processes, coforming processes, spunbonding/meltblowing/spunbonding processes (SMS), spunbonding/meltblowing processes (SM), and bonded carded web processes. For instance, in one embodiment, the nonwoven layer of a laminate moisture barrier of the present invention is an SMS and/or SM material. An SMS material is described in U.S. Pat. No. 4,041,203 to Brock et al., which is incorporated herein in its entirety by reference. Other SMS products and processes are described for example in U.S. Pat. Nos. 5,464,688 to Timmons et al., 5,169,706 to Collier et al., and 4,766,029 to Brock et al., all of which are also incorporated herein in their entireties by reference. Generally, an SMS material will contain a meltblown web sandwiched between two exterior spunbond webs. Such SMS laminates are available from Kimberly-Clark Corporation under marks such as SPUNGUARD and EVOLUTION-brand laminates. The spunbonded layers on the SMS laminates provide durability and the internal meltblown barrier layer provides porosity and additional cloth-like feel. Similar to an SMS laminate, an SM laminate is a spunbond layer laminated to a meltblown layer.

In forming a device of the present invention with a barrier such as a moisture barrier, the barrier can be bonded together with the other layers of the glove in a number of various ways. Thermal bonding, adhesive bonding, ultrasonic bonding, extrusion coating, and the like, are merely examples of various bonding techniques that may be utilized in the present process to attach the barrier to the fibrous layers of the device.

In some embodiments, any of the above layers and/or materials can also be dyed or colored so as to form a base web or barrier having a particular color. For example, in one embodiment, the barrier material 50 can be provided with a colored background.

As described above, the device of the present invention can be made from various components that contain various features. For instance, the device can include a nonelastic component, a elastic component, and a moisture barrier. Further, the device can be made from single layer materials or laminates which, in turn, can be made from various materials and fibers. One particular embodiment of a device made in accordance with the present invention will now be discussed with reference to FIG. 2. In this embodiment, the device 10 includes the first section 20 thermally bonded to the second section 30. The second section 30 is designed for contacting the body of the user, while the first section 20 is made from an elastic laminate for providing the device with form-fitting properties.

The first section 20 can be attached to the second section 30 using various methods. For example, as shown in FIG. 2, the first section 20 can be ultrasonically bonded to the second section 30 along the other edges in order to form a pocket for the insertion of a finger.

Once the first section 20 and the second section 30 are bonded together, excess material can be cut and removed from the device. In general, any suitable cutting method can be used in order to trim away excess material. For example, the material can be cut using a high pressure jet of water referred to as a water knife or can be cut using a conventional mechanical device, such as a cutter or a pair of shears. In one embodiment, the first section 20 and the second section 30 can be simultaneously bonded together and cut from the materials from which they are made. For instance, ultrasonic energy can be used to bond and cut materials in one step.

The dimensions of the device that is formed in accordance with the present invention will depend upon the particular application and purpose for which the device is to be used. For instance, the device can be constructed to fit around the finger of an adult or the finger of a child. Further, the device can also be constructed to fit around two fingers. For most single devices, the glove should have a length of from about 1 inch to about 5 inches and a median flattened width of from about 0.5 inches to about 1.5 inches. When constructed to fit around two fingers, the device can have a median width of from about 0.75 inches to about 2.5 inches, depending on the elasticity of the glove. The device can also be constructed such that it fits over two or more knuckles of the wearer.

In use, the device may be used as only a collection device in which a biological sample is collected using the device. A user wears the device on a finger and uses the device to collect a sample from a body or an object by swabbing, wiping, or other suitable action. The device including the sample is then transferred to a separate diagnostic test kit for analyzing the sample. As examples, the test kit may be one used by a non-health professional in a residential setting, by a health professional in a clinical setting, or as a part of a mail-in diagnostic test.

The hydrophobicity of the materials used in the construction of the device can be an advantage in the collection of biologically-relevant proteins, because proteins tend to readily adsorb onto hydrophobic materials.

In some embodiments of this invention, it would be a desirable attribute of the outer material of the device to readily release the sample, such as the protein exemplified above, once it is placed onto a diagnostic test surface. This can be accomplished by mechanical removal (e.g., rubbing the device onto the test surface), and/or by tailoring the protein adsorption properties of the collection device material of the present invention to the protein adsorption properties in the diagnostic device. If the diagnostic device has a more hydrophobic surface than the collection device, it will likely pick up the protein from the device. This may also be accomplished by liquid extraction (e.g., using water or nonaqueous solvents), or any other suitable method. A similar method would be to use a wash solution, such as a weak surfactant solution (e.g., 0.1 to 1% non-ionic surfactant) to rinse the protein from the collection device and onto the diagnostic surface.

The collection device can also provide stability to the biological sample that is immobilized on its surface during shipping or handling. The addition of some natural fibers, such as cellulose, may be suitable for DNA-based samples or many proteins. In some cases, a coform type of material would be desirable, such that both hydrophobic (e.g., polypropylene) and hydrophilic (e.g., cellulose) materials are present in the collection device. Alternatively, or in addition to the above, a stabilizing agent can be coated on to the fibers of the collection device. Exemplary agents include saccharides (e.g., glucose, sucrose, and trehalose); glycerin; other proteins (e.g., beta casein, serum albumins); and polymers (e.g., polyvinyl alcohol, and polyethylene glycol).

The use of a finger-based device constructed from generally flexible materials increases the dexterity with which a user is able to collect an adequate amount of body fluid, because the user can simply insert their encased finger into or onto the necessary body orifice or part to retrieve a sample that adsorbs onto or absorbs into the device.

Examples of biological samples that may collected with the device include saliva; mucous; lung-based sputum; oral plaque; nasal fluid; tears from the eyes; ear wax; vaginal/cervical fluid or menses; seminal fluid; urine; a blood sample from a cut or self-inflicted puncture or from any other source; a fecal sample; sweat, oils, or cells from the skin; debris from the scalp (e.g., to detect lice); cerebrospinal fluid; amniotic fluid; synovial fluid; serous fluid; and bronchial washings. The device can be used to collect any suitable liquid, solid, or semi-solid substance from a body or an object, including any test subject. The test subject is generally any surface associated with a body or an object to be tested or treated.

In an alternate embodiment, the device may be provided with indicator chemistry that can be used to identify a particular substance in the sample taken. A biological sample is collected using the device. After waiting a sufficient incubation time, the device is placed into a reader for analysis. The device includes the necessary chemistry that causes a detectable change to occur in the presence of the analyte, or targeted substance. This change can be detectable using a reader in cases where it provides greater accuracy or if a quantitative measurement is desired.

In another alternate embodiment, the device again may be provided with indicator chemistry that can be used to identify a particular substance in the sample taken. In this embodiment, however, the indicator can be observed without the use of a reader. A biological sample is collected using the device. After waiting a sufficient incubation time, the device is examined for any visual change that would indicate the presence of a particular substance. Again, the device includes the necessary chemistry that causes a detectable change to occur in the presence of the analyte. This change can be detectable to the naked eye, such as a color change.

The indicator chemistry may include an indicator agent such as methyl red, bromothymol blue, nitrazine, sulfanilamide compounds with acidic buffers, diazonium salts with acidic buffers, glucose oxidase/peroxidase, indoxylcarbonic acid ester modified with a diazonium salt, dichlorobenzene diazonium tetrafluoroborate, tetramethylbenzidine in the presence of peroxide, or methoxybenzene diazonium tetrafluoroborate.

As one example, the collection device may be impregnated with a pH-indicating dye, such as methyl red. Bromothymol blue can be used. The device could be inserted into the vagina for collection of vaginal fluid, and then immediately observed for color change. The user could then determine their vaginal pH, using a color chart that corresponds to pH and comparing the color from the collection device. In this way, the vaginal pH could help screen for certain forms of vaginitis (e.g., bacterial vaginosis and trichomoniasis are associated with elevated vaginal pH>4.5); vaginal pH could also be used to screen premature rupture of membrane as an indicator of imminent or pre-term birth, as well as other conditions associated with abnormal vaginal pH. If desired, a reader could be used for possibly more accurate color comparisons, thereby allowing a precise pH reading to be obtained.

Additional examples for urine-based testing include the use of the following reagents impregnated into the collection device, which is then exposed to urine to monitor a color change:

a) sulfanilamide compounds with acidic buffers to detect nitrites, which are indicative of urinary tract infections

b) diazonium salts with acidic buffers to detect bilirubin, which is indicative of jaundice or other liver diseases

c) glucose oxidase/peroxidase to detect glucose, which is indicative of diabetes or poor diabetic control.

As another example, for the detection of leukocytes or white blood cells, the collection device may be impregnated with a leukocyte-sensitive dye system, such as using an indoxylcarbonic acid ester modified with a diazonium salt, which produces a colored product in the presence of white blood cells. The device would change color (e.g., turn purple) in the presence of leukocytes, which are indicative of infection. In one use, the device could be placed in a urine stream during urination to allow for collection of urine, and then immediately observed for color change. The user could compare the color change from the collection device to a color chart that corresponds to leukocyte levels; high levels would indicate a possible urinary tract infection or other bacterial infection.

For the detection of bilirubin, an indicator of blood, dichlorobenzene diazonium tetrafluoroborate will produce a color. Another indicator for blood is the reaction of tetramethylbenzidine in the presence of peroxide, which will produce a blue color. Finally, for the detection of urobilinogen, an indicator of fecal contamination, methoxybenzene diazonium tetrafluoroborate will produce a color.

Further examples are described in “Textbook of Urinalysis and Body Fluids” by Landy J. McBride, ISBN 0-397-55231-9, which is incorporated herein in its entirety. This textbook includes biological markers that can be found in urine or other body fluids, and the condition that they indicate if found. The examples described herein are presented for the purpose of illustration only and not for limitation.

In another alternate embodiment, the device may be used as an application device in which an indicating substance is applied to the body. In this case, the device includes the necessary chemistry that causes a detectable change to occur in the presence of the analyte. This change can be detectable to the naked eye, such as a color change; or it may be that a reader is desired in cases where it provides greater accuracy or if a quantitative measurement is desired.

In this embodiment, the device leaves a residual agent on the body of the wearer that can react with a target substance to provide a visible indicator of the presence of the target substance. For example, a chemical may be applied to a lesion, in which the chemical may change color to provide an indication of the nature of the lesion. For example, dyes that screen for epithelial cancer may be applied, including the ORATEST-brand screening products of Zila, Inc., and any of the cancer screening technologies described in U.S. Pat. Nos. 5,882,627, 6,086,852, and 6,194,573, and PCT Patent Nos. WO 94/16325 and WO 97/31675. In some cases, ultraviolet light or other light wavelengths may need to be applied to render the indicator visible, or a secondary reagent may need to be applied before or after the residual agent is applied to create a visible change that indicates the presence of the analyte. The secondary reagent can be applied via a wash, a spray, a paste, a pharmaceutical substance, contact with a treated tissue or wipe, including wet wipes, or other suitable method.

In combination with or independent of any of the above approaches, the device may provide further benefits by applying an agent that promotes health, cleanses, refreshes, or serves other useful functions apart from collecting biological materials or indicating the presence of an analyte. For example, analgesics for wounds or sores may be applied, as well as anti-gingivitis medications, anti-microbials, baking powder, vitamin E, any suitable medicament, and so forth. The agent may be impregnated in a nonwoven web, laminated in discrete regions between two nonwoven webs, microencapsulated for release during abrasive action, retained by a soluble binder for release when wet, or manually applied to the product before use (e.g., a toothpaste or dipping solution). A pleasant flavor or fragrance may also be added. Microencapsulation of cleansing agents, antibacterial agents, indicators, dyes, and/or other chemicals in small capsules could then be released slowly during use of the product as the capsules are crushed. As one example, the device would provide several functions in the mouth, because the device could act not just as a means of delivering refreshing or cleaning or antibacterial agents, but also function as a biosensor through incorporation of an indicator, dye, or other reagent that is slowly released during brushing. The same could be true for other body regions as well. See U.S. Pat. No. 6,153,219 for examples of encapsulating foaming or other agents that can be released during brushing.

The device may also be used in a two-step process, with one device used to make a diagnosis or establish the presence of a condition as described herein, and a second device used to apply a beneficial or therapeutic agent to treat or otherwise ameliorate that condition.

Further, in any of the embodiments, all or part of the outer surface of the device may be texturized to assist in sample collection or application.

A disposable collection or application device can be provided with multiple elements for detecting the presence of multiple analytes in a single use. For example, cell-like raised elements could each include a different biosensing element such that a variety of analytes could be detected in saliva during a single cleansing.

In general, the chemical additives described above can be applied to a device of the present invention according to a number of ways known in the art. For example, the additives can be applied to the glove using a saturant system, such as disclosed in U.S. Pat. No. 5,486,381 to Cleveland et al., which is incorporated herein by reference. Moreover, the additives can also be applied by print, roll, blade, spray, spray-drying, foam, brush treating applications, etc., which are all well known in the art. The additives can further be applied as a mixture of molten solids or co-extruded onto the glove. Additionally, in another embodiment, the chemical additives can be impregnated into the material during manufacturing as is well known in the art. It should be understood that when coated onto a glove as described above, the additives can be applied to the base web before or after the base web is stamped or bonded to form a device of the present invention. Furthermore, it should also be understood that, if desired, various additives, solutions, and chemicals can be applied by the consumer to the glove just before use.

Regardless of the mechanism utilized to apply the chemical additives to the glove, the additives can be applied to the glove via an aqueous solution, non-aqueous solution, oil, lotion, cream, suspension, gel, etc. When utilized, an aqueous solution can contain any of a variety of liquids, such as various solvents and/or water. Moreover, the solution can often contain more than one additive. In some embodiments, the additives applied by an aqueous solution or otherwise constitute approximately less than 80% by weight of the device. In other embodiments, the additives can be applied in an amount less than about 50% of the weight of the glove. Moreover, in some embodiments, the additives can also be applied asymmetrically onto the glove to reduce costs and maximize performance of the glove. For instance, in one embodiment, a flat sheet of the base web is asymmetrically contacted with a particular coating agent, and thereafter stamped and bonded to form a device of the present invention, wherein only the surface used to clean teeth is coated with the additives. In another embodiment, the device is stamped and bonded, and thereafter asymmetrically coated with a particular coating agent.

In addition, the disclosed devices could be aseptically sealed for hygienic purposes during storage; in most cases, it would be preferred to have each product individually wrapped and sealed to allow the user to carry only the number of devices needed at one time. Prior to being shipped and sold, the device of the present invention can be placed in various packaging in order to preserve any additives applied to the device or otherwise to maintain the device in a sterile environment. Various packaging materials that can be used include ethylene vinyl alcohol films, film/foil laminates, metalized films, multi-layered plastic films, and the like.

A key advantage that the proposed device offers for the user is isolation and protection from getting in contact with body fluids. Swabs, wipes, or other such collection devices do not offer this same level of protection. Another advantage to the proposed device is the dexterity offered by still being able to use one's finger rather than manipulate a swab; this dexterity allows better sample collection in some cases.

While the invention has been described in conjunction with several specific embodiments, it is to be understood that many alternatives, modifications and variations will be apparent to those skilled in the art in light of the foregoing description.

Accordingly, this invention is intended to embrace all such alternatives, modifications and variations that fall within the spirit and scope of the appended claims.

We claim:

1. A device for capturing a substance, the device comprising:
a generally tubular body including
a generally tubular inner surface defined by a first layer, the inner surface defining a pocket therewithin, the pocket having a distal end and a proximal end, the distal end being generally closed and the proximal end being generally open, the proximal end being configured to allow the insertion of a finger into the pocket through the proximal end, and
a generally tubular outer surface generally disposed radially outwardly from the inner surface, wherein a portion of the outer surface is adapted to capture the substance.

2. The device of claim 1, wherein the first layer includes a barrier material.

3. The device of claim 2, wherein the barrier material is breathable to water vapor.

4. The device of claim 1, further comprising a second layer coupled to the first layer.

5. The device of claim 4, wherein the second layer includes at least two layers of material such that the second layer constitutes a laminate.

6. The device of claim 4, wherein the second layer includes a nonwoven material.

7. The device of claim 6, wherein the nonwoven material is a thermoplastic material.

8. The device of claim 6, wherein the nonwoven material is a polypropylene material.

9. The device of claim 6, wherein the nonwoven material is a hydrophobic material.

10. The device of claim 6, wherein the nonwoven material is a hydrophilic material.

11. The device of claim 6, wherein the nonwoven material is a mixture of hydrophobic and hydrophilic materials.

12. The device of claim 6, wherein the nonwoven material is selected from the group consisting of spunbonded fiber materials, meltblown fiber materials, spunbonded/meltblown/spunbonded fiber materials, spunbonded/meltblown fiber materials, coform, and bonded carded materials.

13. The device of claim 6, wherein the nonwoven material comprises an elastic component.

14. The device of claim 13, wherein the elastic component includes a fibrous material.

15. The device of claim 13, wherein the elastic component includes a film.

16. The device of claim 1, wherein the portion is adapted to retain the substance.

17. The device of claim 1, wherein the portion is adapted to release the substance.

18. The device of claim 1, wherein the portion is adapted to release the substance to a biosensor for testing.

19. The device of claim 1, wherein the substance is an indicator agent.

20. The device of claim 19, further comprising a second portion of the outer surface adapted to capture a second substance.

21. The device of claim 20, wherein the second substance is a second indicator agent.

22. The device of claim 20, wherein the second substance is a beneficial agent.

23. The device of claim 19, wherein the portion is adapted to deposit the indicator agent on a test subject.

24. The device of claim 23, wherein the test subject is associated with a body.

25. The device of claim 19, wherein the indicator agent is adapted to indicate pH.

26. The device of claim 25, wherein the pH indication is used to detect premature rupture of membrane.

27. The device of claim 25, wherein the pH indication is used to detect bacterial or trichomonal vaginal infections.

28. The device of claim 19, wherein the indicator agent is selected from the group consisting of: methyl red, bromothymol blue, nitrazine, sulfanilamide compounds with acidic buffers, diazonium salts with acidic buffers, glucose oxidase/peroxidase, indoxylcarbonic acid ester modified with a diazonium salt, dichlorobenzene diazonium tetrafluoroborate, tetramethylbenzidine in the presence of peroxide, and methoxybenzene diazonium tetrafluoroborate.

29. The device of claim 1, further comprising an elastic component.

30. The device of claim 29, wherein the elastic component includes a fibrous material.

31. The device of claim 29, wherein the elastic component includes a film.

32. The device of claim 1, wherein the barrier material includes a moisture barrier, the moisture barrier being substantially impermeable to liquids when contacted therewith.

33. The device of claim 32, wherein the moisture barrier comprises a plastic film.

34. The device of claim 33, wherein the plastic film is a microporous film.

35. The device of claim 32, wherein the moisture barrier comprises a multilayered laminate.

36. The device of claim 35, wherein one of the layers of the moisture barrier comprises a nonwoven web of fibrous material.

37. The device of claim 35, wherein one of the layers of the moisture barrier comprises a vapor-permeable film.

38. The device of claim 1, wherein the substance is selected from the group consisting of: saliva, mucous, lung-based sputum, oral plaque, nasal fluid, tears, ear wax, vaginal fluid, cervical fluid, menses, seminal fluid, urine, blood, feces, sweat, skin oils, skin cells, scalp debris, cerebrospinal fluid, amniotic fluid, synovial fluid, serous fluid, and bronchial washings.

39. The device of claim 1, wherein the substance is a beneficial agent, and wherein a portion of the device is adapted to deposit the beneficial agent on a test subject.

40. The device of claim 39, wherein the beneficial agent is selected from the group consisting of: medicaments, diaper rash ointments, alcohols, anesthetics, analgesics, facial make-up removal agents, anti-microbials, antibacterials, baking powder, moisturizing agents, lubricants, vitamins, and nutrients.

41. A method for collecting a sample from a test subject, the method comprising:
providing a device adapted to capture and retain the sample, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface;
inserting a finger into the pocket; and
contacting the sample with the device.

42. The method of claim 41, further comprising:
providing a second device adapted to capture and release an agent, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface;
inserting a finger into the pocket; and
contacting the sample with the second device such that the agent is released.

43. The method of claim 41, wherein the contacting act includes contacting a sample selected from the group consisting of: saliva, mucous, lung-based sputum, oral plaque, nasal fluid, tears, ear wax, vaginal fluid, cervical fluid, menses, seminal fluid, urine, blood, feces, sweat, skin oils, skin cells, scalp debris, cerebrospinal fluid, amniotic fluid, synovial fluid, serous fluid, and bronchial washings.

44. The method of claim 41, wherein the providing act includes providing a device with an interior layer including a barrier material, and wherein the barrier material is breathable to water vapor.

45. The method of claim 44, wherein the barrier material includes a moisture barrier, the moisture barrier being substantially impermeable to liquids when contacted therewith.

46. A method for analyzing a sample, the method comprising:
providing a device adapted to capture and retain the sample, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface;
contacting the substance to be sampled with the device; and
analyzing the device using a reader.

47. The method of claim 46, further comprising:
providing a second device adapted to capture and release an agent, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface;
inserting a finger into the pocket; and
contacting the test subject with the second device such that the agent is released.

48. The method of claim 46, wherein the contacting act includes contacting a substance selected from the group consisting of: saliva, mucous, lung-based sputum, oral plaque, nasal fluid, tears, ear wax, vaginal fluid, cervical fluid, menses, seminal fluid, urine, blood, feces, sweat, skin oils, skin cells, scalp debris, cerebrospinal fluid, amniotic fluid, synovial fluid, serous fluid, and bronchial washings.

49. The method of claim 46, wherein the providing act includes providing a device with an interior layer including a barrier material, and wherein the barrier material is breathable to water vapor.

50. The method of claim 49, wherein the barrier material includes a moisture barrier, the moisture barrier being substantially impermeable to liquids when contacted therewith.

51. A method for analyzing a sample, the method comprising:
providing a device adapted to capture and retain the sample, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface including an indicator agent;
contacting the substance to be sampled with the device; and
observing the reaction of the sample with the indicator agent on the device.

52. The method of claim 51, further comprising:
providing a second device adapted to capture and release an agent, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface;
inserting a finger into the pocket; and
contacting the test subject with the second device such that the agent is released.

53. The method of claim 51, wherein the contacting act includes contacting a substance selected from the group consisting of: saliva, mucous, lung-based sputum, oral plaque, nasal fluid, tears, ear wax, vaginal fluid, cervical fluid, menses, seminal fluid, urine, blood, feces, sweat, skin oils, skin cells, scalp debris, cerebrospinal fluid, amniotic fluid, synovial fluid, serous fluid, and bronchial washings.

54. The method of claim 51, wherein the providing act includes providing a device with an interior layer including a barrier material, and wherein the barrier material is breathable to water vapor.

55. The method of claim 54, wherein the barrier material includes a moisture barrier, the moisture barrier being substantially impermeable to liquids when contacted therewith.

56. The method of claim 51, wherein the observing act includes observing the reaction without electromechanical assistance.

57. The method of claim 51, wherein the observing act includes observing the reaction with the aid of a light source.

58. The method of claim 51, wherein the observing act includes observing the reaction using a reader.

59. A method for analyzing a test subject, the method comprising:
providing a device adapted to capture and release an indicator agent, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface including the indicator agent;
contacting the test subject to be analyzed with the device, such that at least a portion of the indicator agent is released to the test subject; and
observing the reaction of the test subject with the indicator agent.

60. The method of claim 59, further comprising:
providing a second device adapted to capture and release an agent, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface;
inserting a finger into the pocket; and
contacting the test subject with the second device such that the agent is released.

61. The method of claim 59, wherein the providing act includes providing a device with an interior layer including a barrier material, and wherein the barrier material is breathable to water vapor.

62. The method of claim 61, wherein the barrier material includes a moisture barrier, the moisture barrier being substantially impermeable to liquids when contacted therewith.

63. The method of claim 61, wherein the observing act includes observing the reaction without electromechanical assistance.

64. The method of claim 61, wherein the observing act includes observing the reaction with the aid of a light source.

65. The method of claim 61, wherein the observing act includes observing the reaction using a reader.

66. A method for applying a substance to a test subject, the method comprising:
providing a device adapted to capture and release the substance, wherein the device includes a generally tubular body including a generally tubular inner surface defined by an interior layer, the inner surface defining a pocket therewithin, the pocket having a distal end and a proximal end, the distal end being generally closed and the proximal end being generally open, the proximal end being configured to allow the insertion of a finger into the pocket through the proximal end, and a generally tubular outer surface, wherein the outer surface includes the substance;
contacting the test subject with the device such that at least a portion of the substance is released from the device and deposited on the test subject.

67. The method of claim 66, wherein the providing act includes providing an indicator agent.

68. The method of claim 67, wherein the indicator agent is selected from the group consisting of: methyl red, bromothymol blue, nitrazine, sulfanilamide compounds with acidic buffers, diazonium salts with acidic buffers, glucose oxidase/peroxidase, indoxylcarbonic acid ester modified with a diazonium salt, dichlorobenzene diazonium tetrafluoroborate, tetramethylbenzidine in the presence of peroxide, and methoxybenzene diazonium tetrafluoroborate.

69. The method of claim 66, wherein the providing act includes providing a beneficial agent.

70. The method of claim 69, wherein the beneficial agent is selected from the group consisting of: medicaments, diaper rash ointments, alcohols, anesthetics, analgesics, facial make-up removal agents, anti-microbials, antibacterials, baking powder, moisturizing agents, lubricants, vitamins, and nutrients.

71. A device for capturing a substance, the device comprising:
a generally tubular body including
a generally tubular inner surface defined by a first layer, the first layer, wherein the barrier material includes a moisture barrier that is substantially impermeable to liquids when contacted therewith, the inner surface defining a pocket therewithin, the pocket having a distal end and a proximal end, the distal end being generally closed and the proximal end being generally open, the proximal end being configured to allow the insertion of a finger into the pocket through the proximal end, and
a generally tubular outer surface generally disposed radially outwardly from the inner surface, wherein a portion of the outer surface is adapted to capture the substance.

72. A diagnostic kit comprising:
a collection device including a generally tubular body, the body including
a generally tubular inner surface defined by a first layer, the inner surface defining a pocket therewithin, the pocket having a distal end and a proximal end, the distal end being generally closed and the proximal end being generally open, the proximal end being configured to allow the insertion of a finger into the pocket through the proximal end, and
a generally tubular outer surface generally disposed radially outwardly from the inner surface, wherein a portion of the outer surface is adapted to capture the substance; and
an indicator device adapted to generate a diagnosis using the substance from the collection device.

73. A sample collection system comprising:
a first collection device to collect a first substance, wherein the first collection device includes a generally tubular body, the body including
a generally tubular inner surface defined by a first layer, the inner surface defining a pocket therewithin, the pocket having a distal end and a proximal end, the distal end being generally closed and the proximal end being generally open, the proximal end being configured to allow the insertion of a finger into the pocket through the proximal end, and
a generally tubular outer surface generally disposed radially outwardly from the inner surface, wherein a portion of the outer surface is adapted to capture the substance; and
a second collection device to collect a second substance.

74. A sample collection system comprising:
a first a collection device including a generally tubular body, the body including
a generally tubular inner surface defined by a first layer, the inner surface defining a pocket therewithin, the pocket having a distal end and a proximal end, the distal end being generally closed and the proximal end being generally open, the proximal end being configured to allow the insertion of a finger into the pocket through the proximal end, and
a generally tubular outer surface generally disposed radially outwardly from the inner surface, wherein a portion of the outer surface is adapted to capture the substance; and
a second collection device larger than the first collection device.
